(19) United States
(12) Patent Application Publication
(10) Pub. No.: US 2024/0166654 A1
(43) Pub. Date: May 23, 2024

(54) RUXOLITINIB CRYSTAL AND PHARMACEUTICAL COMPOSITION THEREOF

(71) Applicant: ZHEJIANG AUSUN PHARMACEUTICAL CO., LTD., Linhai (CN)

(72) Inventors: Liangang SHEN, Linhai (CN); Xianfeng WU, Linhai (CN); Ronghua ZHU, Linhai (CN); Zhiguo ZHENG, Linhai (CN)

(21) Appl. No.: 18/505,264

(22) Filed: Nov. 9, 2023

(30) Foreign Application Priority Data
Nov. 11, 2022 (CN) 202211411151.7

(51) Int. Cl. C07D 487/04; A61K 31/519

(52) U.S. Cl. CPC C07D 487/04; A61K 31/519

(57) ABSTRACT

The invention relates to ruxolitinib crystal and a pharmaceutical composition thereof. In particular, the invention provides ruxolitinib dihydrate crystal and a preparation method thereof, and ruxolitinib free base in amorphous form and a preparation method thereof, and a pharmaceutical composition comprising ruxolitinib dihydrate crystal or ruxolitinib free base in amorphous form. The crystal, amorphous form and pharmaceutical composition of the invention have simple preparation method, high product yield, and excellent crystal stability, hygroscopicity and processability, and thus are suitable for industrial production.

### CROSS-REFERENCE TO RELATED APPLICATIONS

This application is a U.S. nonprovisional patent application filed under 35 U.S.C. § 111(a) which claims priority to, and the benefit of, Chinese Patent Application No. 202211411151.7, filed on Nov. 11, 2022, the contents of which are hereby incorporated by reference in its entirety.

### TECHNICAL FIELD

The invention belongs to the field of pharmaceutical chemistry. In particular, the invention relates to crystal and amorphous form of ruxolitinib free base, a preparation method thereof and their use in pharmaceutical compositions.

### BACKGROUND

Ruxolitinib phosphate is an orally administrable selective JAK1/JAK2 kinase inhibitor developed by Incyte Corporation of the United States. It is the first drug (trade name JAKAF®) approved by FDA of the United States for the treatment of myelofibrosis in November 2011. It was approved for marketing in EU by Novartis in August 2012 and approved for marketing in China in March 2017.

As a highly selective, minimally toxic side effects, and well-tolerated oral therapy, ruxolitinib phosphate applies to the treatment of moderate or high-risk myelofibrosis (MF), including primary MF, MF after polycythemia vera, and MF after primary thrombocythemia. In recent years, its indications have been continuously expanded, showing good application prospects. Its chemical name is (R)-3-(4-(7H-pyrrolo[2,3-d]pyrimidin-4-yl)-1H-pyrazol-1-yl)-3-cyclopentyl propionitrile phosphate, and its chemical structural formula is as follows:

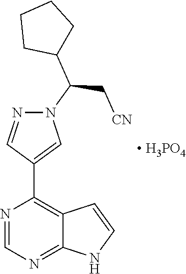

WO2007070514A1 discloses the preparation of ruxolitinib free base in (R) and (S) configurations by HPLC, but there is no description of its purity and physical state.

WO2008157208A2 discloses the preparation of ruxolitinib phosphate, sulfate and maleate, but does not describe the physical state of ruxolitinib free base either.

Organic Letters, 2009, 11(9), 1999-2002 and patent application WO2010083283A2 respectively disclose the preparation method and physical state of ruxolitinib free base, and its physical state is white or light yellow foamy solid. After repeated experiments and determination, the foamy solid compound is amorphous form. In addition, Organic Letters, 2009, 11(9), 1999-2002 also discloses that ruxolitinib free base obtained after column chromatography is oil, which is solidified at room temperature under vacuum, but there is no description of its melting point and crystalline form.

Angew. Chem. Int. Ed. 2015, 54, 7149-7153 discloses the preparation method of ruxolitinib free base and its physical state as colorless foam, which crystallizes by standing to obtain solid with melting point of 88° C., but there is no further description of its crystalline form.

### BRIEF SUMMARY OF THE INVENTION

The invention has for the first time discovered a new crystalline form of ruxolitinib free base, which is simple to prepare and excellent in stability, solubility, hygroscopicity and processability, thus providing suitable raw materials for dry granulation or compression method development of pharmaceutical preparations. The invention also relates to a use of the crystalline form in the pharmaceutical composition and a pharmaceutical composition comprising the crystalline form and one or more pharmaceutically acceptable excipients or carriers.

### DETAILED DESCRIPTION OF THE INVENTION

In a first aspect, the invention provides ruxolitinib dihydrate of Formula I.

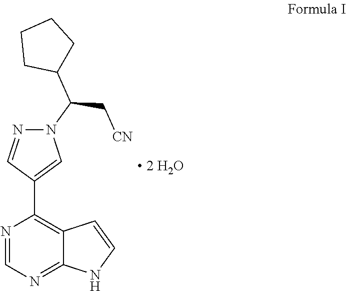 [SMILES: N#CC[C@H](C1CCCC1)n1cc(-c2ncnc3[nH]ccc23)cn1 ; "· 2 H2O"]

In one embodiment, the compound of formula I is in crystalline form characterized in that its X-ray powder diffraction pattern has characteristic diffraction peaks expressed in 2θ diffraction angle at 6.92±0.2°, 19.02±0.2°, 22.62±0.2°, 23.12±0.2° and 24.66±0.2°.

In a preferred embodiment, the compound of formula I is in crystalline form, characterized in that its X-ray powder diffraction pattern has characteristic diffraction peaks expressed in 2θ diffraction angle at 6.92±0.2°, 10.54±0.2°, 11.54±0.2°, 15.42±0.2°, 19.02±0.2°, 22.62±0.2°, 23.12±0.2° and 24.66±0.2°.

In a more preferred embodiment, the compound of formula I is in crystalline form, characterized in that its X-ray powder diffraction pattern has characteristic diffraction peaks expressed in 2θ diffraction angle at 6.92±0.2°, 10.54±0.2°, 11.54±0.2°, 15.42±0.2°, 19.02±0.2°, 20.78±0.2°, 22.62±0.2°, 23.12±0.2°, 24.66±0.2° and 25.76±0.2°.

In a more preferred embodiment, the compound of formula I is in crystalline form, characterized in that its X-ray powder diffraction pattern has characteristic diffraction peaks expressed in 2θ diffraction angle at 6.92±0.2°, 10.54±0.2°, 11.54±0.2°, 15.08±0.2°, 15.42±0.2°, 16.20±0.2°, 19.02±0.2°, 20.78±0.2°, 21.78±0.2°, 22.62±0.2°, 22.86±0.2°, 23.12±0.2°, 24.66±0.2°, 25.76±0.2° and 27.50±0.2°.

The crystalline form of compound of formula I, when Cu-Kα (λ=1.548162 Å) radiation, tube voltage 40 kV, tube current 15 mA, scan speed 5°/min, step width 0.02°, DS (slit) 0.625, θ-2θ continuous scanning with scan range 3-40° (20) are used, has an X-ray powder diffraction pattern as shown in FIG. 1. Among them, the main characteristic diffraction profile expressed in 2θ diffraction angle is shown in Table 1.

TABLE 1
Main characteristic diffraction profile of compound of formula I

| Diffraction | | |
|---|---|---|
| angle 2θ (°) | Interplanar spacing d (Å) | Relative intensity I/I0 (%) |
| 6.92 | 12.77 | 55.8 |
| 7.46 | 11.85 | 2.3 |
| 10.54 | 8.39 | 41.3 |
| 11.18 | 7.91 | 11.0 |
| 11.54 | 7.67 | 40.6 |
| 12.62 | 7.01 | 21.0 |
| 12.94 | 6.84 | 24.5 |
| 13.84 | 6.40 | 13.5 |
| 14.62 | 6.06 | 21.8 |
| 15.08 | 5.88 | 27.5 |
| 15.42 | 5.75 | 50.5 |
| 16.20 | 5.47 | 28.3 |
| 17.72 | 5.01 | 10.5 |
| 18.08 | 4.91 | 12.4 |
| 19.02 | 4.67 | 79.7 |
| 20.04 | 4.43 | 11.2 |
| 20.78 | 4.28 | 34.2 |
| 21.02 | 4.23 | 11.8 |
| 21.78 | 4.08 | 25.8 |
| 22.62 | 3.93 | 100.0 |
| 22.86 | 3.89 | 28.4 |
| 23.12 | 3.85 | 71.2 |
| 23.90 | 3.72 | 13.8 |
| 24.06 | 3.70 | 11.1 |
| 24.66 | 3.61 | 54.0 |
| 25.76 | 3.46 | 35.2 |
| 26.04 | 3.42 | 16.4 |
| 27.28 | 3.27 | 20.8 |
| 27.50 | 3.24 | 29.6 |
| 29.64 | 3.01 | 14.7 |
| 30.06 | 2.97 | 23.7 |

The single crystal structure data of compound of formula I of the invention is shown in Table 2, and the non-hydrogen atom coordinates and various temperature factors of the molecule of compound of formula I are shown in Table 3.

The crystal structure analysis results show that the crystal of compound of formula I belongs to orthorhombic system, space group P2₁2₁2₁ (19#), and the unit cell parameters are: α=9.8770(3) Å, b=15.0030(4) Å, c=23.5866(6) Å, V=3495.17(17) Å³, Z=8, molecular formula: C₁₇H₁₈N₆·2(H₂O), the molecular structural formula is shown in Formula I (system naming ((3R)-3-cyclopentyl-3-[4-(7H-pyrrolo[2,3-d]pyrimidin-4-yl)pyrazol-1-yl]propionitrile)·2H₂O). The smallest asymmetric unit contains two ruxolitinib molecules and four water molecules, i.e., ruxolitinib dihydrate crystalline form. The atom number is shown in FIG. 2. The water molecules in the structure form hydrogen bonds with amino nitrogen atoms on pyrazole ring and pyrimidine ring in ruxolitinib molecule respectively. The chiral carbon atoms (C10, C10A) in the two ruxolitinib molecules have the same configuration, both of which are in the “R” configuration. The molecular configuration is shown in FIG. 3. Two ruxolitinib molecules are bridged by four water molecules to form a dimer structure (see FIG. 4). This dimer forms a three-dimensional stacked structure through hydrogen bonding (see FIG. 5).

The DSC curve of the crystal of compound of formula I of the invention is shown in FIG. 6, showing a melting point of 67.1° C.

The infrared spectrum of the crystal of compound of formula I of the invention is shown in FIG. 7.

TABLE 2
Single crystal structure data and structure correction
data of the molecule of compound of formula I

| Shape, color, size of crystal | Block, colorless and transparent; 0.42 mm × 0.25 mm × 0.15 | |
|---|---|---|
| | mm | |
| Crystal system | Orthorhombic | |
| Space group | P2₁2₁2₁ | |
| Cell parameters | a = 9.8770(3)Å, b = 15.0030(4)Å, c = 23.5866(6)Å, | |
| Cell volume | V = 3495.17(17)Å³ | |
| Number of molecules per unit | 8 | |
| cell (Z) | | |
| Molecular weight (Mr) | 342.40 | |
| Calculated density (Dc) | 1.301 | g/cm³ |
| Absorption coefficient (μ) | 0.730 | mm⁻¹ |

| | |
|---|---|
| F(000) | 1456 |
| Final R factor | R₁ = 0.0319, wR₂ = 0.0865 |
| Goodness-of-fit on F2 (S) | 1.054 |
| Shift/su_max (δ/σ) | 0.001 |
| Residual | 0.251 e-/Å³(max), −0.221 e-/Å³(min) |
| Absolute configuration | ad |
| confirmation method | |
| Flack value | 0.03(4) |

Information on diffraction data collection and structural analysis:

| X-ray type and wavelength | Cu Kα, λ = 1.54178 Å | |
|---|---|---|
| Total diffraction data/ | 31693/6384 | |
| independent diffraction | | |
| I greater than 2sigma(I) | 6303 | |
| Data integrity | 99.8% | |
| Corrected variables/number | 475/8 | |
| of constraints | | |
| Structure correction manner | Full matrix least squares correction based on F² | |
| Temperature of diffraction | 170 | K |

data collection

TABLE 3
Atomic coordinates and temperature factors of non-hydrogen atoms

| Atoms | x(*10⁴) | y(*10⁴) | z(*10⁴) | U(eq) (Å²*10³) |
|---|---|---|---|---|
| O1 | 5708.2(16) | 5950.1(11) | 4535.6(7) | 32.6(3) |
| O2 | 5922.5(18) | 6039.8(12) | 3359.9(7) | 40.7(4) |
| O3 | 2948(2) | 3704.5(11) | 1526.7(7) | 39.3(4) |
| O4 | 2319.8(17) | 2245.5(11) | 2221.3(7) | 35.9(4) |
| N1 | 572.4(19) | 7845.7(12) | 4584.8(7) | 29.2(4) |
| N2 | 2900.5(19) | 7911.3(12) | 4305.4(7) | 27.9(4) |
| N3 | 3433.2(17) | 6971.4(12) | 3509.6(7) | 25.7(4) |
| N4 | 1976.6(18) | 5183.6(11) | 2225.6(7) | 26.1(4) |
| N5 | 674.0(18) | 5466.7(11) | 2281.7(7) | 23.5(3) |
| N6 | −988(2) | 3501.7(15) | 2849.2(10) | 43.9(5) |
| C1 | −569(2) | 7413.8(14) | 4383.3(9) | 29.9(4) |
| C2 | −269(2) | 6932.2(13) | 3913.2(9) | 25.7(4) |
| C3 | 1145(2) | 7052.5(12) | 3813.9(8) | 20.1(4) |
| C4 | 1620(2) | 7632.1(13) | 4243.9(8) | 23.2(4) |
| C5 | 3740(2) | 7541.5(15) | 3930.6(9) | 28.6(4) |
| C6 | 2125(2) | 6733.1(12) | 3434.5(8) | 20.4(4) |
| C7 | 1788(2) | 6159.3(13) | 2954.3(8) | 21.8(4) |
| C8 | 529(2) | 6048.0(12) | 2709.2(8) | 23.1(4) |
| C9 | 2651(2) | 5604.5(13) | 2634.1(8) | 24.8(4) |
| C10 | −374(2) | 5188.4(13) | 1877.4(8) | 24.7(4) |
| C11 | −458(2) | 4167.4(14) | 1852.6(9) | 29.6(5) |
| C12 | −744(2) | 3779.4(14) | 2410.9(10) | 31.5(5) |
| C13 | −130(2) | 5598.8(14) | 1294.2(9) | 28.3(5) |
| C14 | −1268(3) | 5399.4(17) | 868.0(10) | 37.4(5) |
| C15 | −1209(4) | 6158(2) | 443.3(14) | 67.6(10) |
| C16 | −367(4) | 6895.0(18) | 694.9(12) | 52.3(7) |
| C17 | −85(3) | 6621.2(16) | 1306.3(11) | 43.1(6) |
| N1A | −890(2) | 5501.6(13) | 4999.4(8) | 32.0(4) |
| N2A | 1342(2) | 5855.5(12) | 5325.6(8) | 29.5(4) |
| N3A | 3109.1(18) | 5253.0(12) | 4739.9(7) | 26.6(4) |
| N4A | 3132.5(18) | 3388.8(12) | 3186.9(7) | 26.6(4) |
| N5A | 4343.6(17) | 3682.8(11) | 3388.4(7) | 22.5(3) |
| N6A | 6132(3) | 4681.4(18) | 2019.9(11) | 58.0(7) |
| C1A | −1417(2) | 5016.1(16) | 4559.0(11) | 34.6(5) |
| C2A | −404(2) | 4653.6(14) | 4238.4(10) | 29.8(4) |
| C3A | 834(2) | 4942.9(13) | 4489.7(8) | 24.3(4) |
| C4A | 481(2) | 5472.3(13) | 4966.9(9) | 27.4(4) |
| C5A | 2624(2) | 5710.9(14) | 5184.7(9) | 29.8(5) |
| C6A | 2220(2) | 4848.3(13) | 4387.9(8) | 22.3(4) |
| C7A | 2816(2) | 4324.6(13) | 3930.2(8) | 22.7(4) |
| C8A | 4192(2) | 4234.3(13) | 3829.9(8) | 23.6(4) |
| C9A | 2215(2) | 3785.5(14) | 3512.4(9) | 26.0(4) |
| C10A | 5643(2) | 3306.0(14) | 3195.2(8) | 25.4(4) |
| C11A | 5648(2) | 3196.9(14) | 2551.7(9) | 29.0(4) |
| C12A | 5881(2) | 4034.7(17) | 2251.3(10) | 35.6(5) |
| C13A | 5997(2) | 2431.2(14) | 3491.7(9) | 31.1(5) |
| C14A | 6273(3) | 2517.9(18) | 4126.2(11) | 43.5(6) |
| C15A | 6138(4) | 1562(2) | 4351.1(12) | 59.5(8) |
| C16A | 5487(4) | 1013.0(19) | 3888.2(14) | 58.7(8) |
| C17A | 4978(3) | 1682.3(16) | 3450.0(11) | 41.1(6) |

In a second aspect, the invention provides a preparation method of crystal of compound of formula I, comprising:

Ruxolitinib free base is added in an organic solvent and dissolved, then purified water is added dropwise to crystallize, to obtain the crystal of compound of formula I.

In a preferred embodiment, the ruxolitinib free base is added in an organic solvent and dissolved. The reaction temperature is controlled to be 10 to 50° C. The purified water is added dropwise. Then the internal temperature is controlled to be 10 to 40° C. and it is stirred to precipitate the crystal, to obtain the crystal of compound of formula I.

In a preferred embodiment, the organic solvent means a solvent capable of dissolving ruxolitinib free base, such as methanol, ethanol, n-propanol, isopropanol, n-butanol, acetone, diethyl ether, isopropyl ether, methyl tert-butyl ether, tetrahydrofuran, 1,4-dioxane, acetonitrile, dichloromethane, chloroform, toluene, chlorobenzene, hexane, heptane, DMF (dimethyl formamide), DMAC (dimethylacetamide), DMSO (dimethyl sulfoxide), NMP (N-methylpyrrolidone), methyl acetate, ethyl acetate, isopropyl acetate, or a mixture of two or more thereof.

In a more preferred embodiment, ruxolitinib free base is added in an organic solvent and dissolved. The reaction temperature is controlled to be 10 to 50° C. The purified water is added dropwise, the system becomes turbid at this time. The internal temperature is controlled to be 10 to 40° C., and it is stirred for 1 to 6 h. A large amount of pale yellow to off-white solid is precipitated. Then the temperature is reduced to −5 to 15° C., and it is stirred for 1 to 2 h, filtered and dried, to obtain the crystal of compound of formula I.

In preparing the crystal of compound of formula I, in order to facilitate rapid crystallization, additional crystal seeds prepared according to the above method can be added to induce crystallization.

The ruxolitinib raw material used may be crude, such as oil, foam or non-viscous gel, or amorphous form as described herein.

The drying may be drying under vacuum. The drying temperature may be 10 to 50° C., preferably 25 to 45° C. The drying time is 3 to 24 h.

In a third aspect, the invention provides amorphous form of ruxolitinib free base and a preparation method thereof.

The invention provides amorphous form of ruxolitinib free base in solid, characterized in that when Cu-Kα (λ=1.548162 Å) radiation, tube voltage 40 kV, tube current 15 mA, scan speed 5°/min, step width 0.02°, DS (slit) 0.625, θ-2θ continuous scanning with scan range 3-40° (2θ) are used, has an X-ray powder diffraction pattern as shown in FIG. 8.

X-ray powder diffraction pattern shows that the solid is in amorphous form. The infrared spectrum of amorphous form is shown in FIG. 9. The DSC pattern of amorphous form is shown in FIG. 10 and has a melting point of 66.14° C.

The invention also provides a preparation method of amorphous form of ruxolitinib free base, which comprises the following steps: ruxolitinib free base raw material is added in an organic solvent and dissolved, antisolvent is added to crystallize, to obtain amorphous form of ruxolitinib free base in solid.

In a preferred embodiment, the preparation method of amorphous form of ruxolitinib free base comprises the following steps: ruxolitinib free base raw material is added in an organic solvent and dissolved, antisolvent is added, the temperature is controlled to crystallize, filtered to obtain amorphous from of ruxolitinib free base in solid.

The ruxolitinib raw material may be crude, such as oil, foam or non-viscous gel, or may be crystal of compound of formula I.

The organic solvent is selected from ethyl formate, methyl acetate, ethyl acetate, isopropyl acetate, diethyl ether, isopropyl ether, methyl tert-butyl ether, tetrahydrofuran, 1,4-dioxane, acetone, dichloromethane, toluene, xylene, chlorobenzene, or a mixture of two or more thereof.

The antisolvent is selected from pentane, n-pentane, neopentane, hexane, n-hexane, cyclohexane, methylcyclohexane, n-heptane, or a mixture of two or more thereof.

The crystallization temperature is −10 to 20° C., preferably −5 to 15° C., most preferably 0 to 10° C.

In another embodiment, the invention provides a preparation method of amorphous form of ruxolitinib free base, comprising: compound of formula I of the invention is dried under vacuum to lose crystal water to obtain amorphous form of ruxolitinib free base.

The drying temperature is 30 to 65° C., preferably 35 to 60° C., most preferably 45 to 55° C. The drying under vacuum has vacuum degree≤−0.1 Mpa. The drying time is 3 to 24 h, preferably 8 to 15 h.

The invention also relates to amorphous form of ruxolitinib free base in solid obtained by the preparation method described above.

In a fourth aspect, the invention provides a pharmaceutical composition comprising ruxolitinib and one or more pharmaceutically acceptable excipients or carriers. The ruxolitinib can be selected from crystal of compound of formula I or amorphous form of ruxolitinib free base.

The pharmaceutical composition of the invention may be prepared according to the methods commonly used in the art and they may be suitable for gastrointestinal administration (e.g., oral or rectal administration) or parenteral administration to a mammal, including a human. The composition comprises therapeutically effective amount of ruxolitinib used in combination with at least one pharmaceutically acceptable excipient or carrier. The typical oral preparations include tablets, capsules, syrups, elixirs, and suspensions.

Th pharmaceutically acceptable excipients or carriers suitable for use in the invention include, but are not limited to, diluents, fillers, disintegrants, glidants, lubricants, binders, colorants, and combinations thereof, provided that they are chemically inert and thus do not adversely affect the active ingredient. The amount of each excipient or carrier in the solid preparation may vary within conventional scope in the art. Examples of typical pharmaceutically acceptable excipients or carriers are: sugars such as lactose, sucrose, mannitol and sorbitol; starches, such as corn starch, tapioca starch and potato starch; celluloses and derivatives thereof, such as microcrystalline cellulose, sodium carboxymethyl cellulose, hydroxypropyl cellulose, hydroxypropyl methyl cellulose, ethyl cellulose and methyl cellulose; calcium phosphates, such as dicalcium phosphate and tricalcium phosphate; sodium sulfate; calcium sulfate; polyvinylpyrrolidone; povidone K30; sodium carboxymethyl starch; polyvinyl alcohol; stearic acid; alkaline earth metal salts of stearic acid, such as magnesium stearate and calcium stearate; colloidal silica; vegetable oils, such as peanut oil, cottonseed oil, sesame oil, olive oil and corn oil; nonionic, cationic and anionic surfactants; ethylene glycol polymers; β-cyclodextrin; fatty alcohols; and grain hydrolyzed solids, as well as other nontoxic compatible fillers, binders, disintegrants, buffers, preservatives, antioxidants, lubricants, colorants and other excipients commonly used in pharmaceutical preparation.

Pharmaceutical preparation for gastrointestinal or parenteral administration may be in unit dosage form such as coated tablets, tablets, capsules or suppositories and ampoules. They may be prepared according to the methods commonly used in the art, for example by conventional mixing, granulation, coating, dissolution or freeze drying. Therefore, the pharmaceutical composition for oral administration can be obtained by: mixing ruxolitinib with solid excipients and optionally granulating the mixture obtained above, followed by optionally adding other excipients, and forming the mixture or granules into tablets or cores of coated tablets.

In a preferred embodiment, the composition of pharmaceutical composition comprises ruxolitinib, an organic acid and other excipients or carriers.

The ruxolitinib can be selected from crystal of compound of formula I or amorphous form of ruxolitinib free base described above. The organic acid can be selected from one or more of malonic acid, maleic acid, fumaric acid, citric acid, tartaric acid and malic acid.

Said other excipients or carriers may include microcrystalline cellulose, lactose monohydrate and povidone K30.

The invention also provides a preparation method of the composition, comprising: ruxolitinib and an organic acid are uniformly mixed in a certain proportion, other excipients or carriers are added, stirred and mixed uniformly, and then dry tableted or granulated. The ruxolitinib can be selected from crystal of compound of formula I or amorphous form of ruxolitinib free base described above.

An advantageous effects of the invention are to provide ruxolitinib free base dihydrate crystal as well as amorphous form. The crystal is excellent in stability, solubility, hygroscopicity and processability, thus providing suitable raw materials for dry granulation or tableting method development of pharmaceutical preparations. Moreover, the preparation method of amorphous form is simple, the obtained product has high yield, good purity and stable property, is suitable for industrial production, and provides suitable raw materials and methods for dry granulation or tableting of drugs.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a XRPD pattern of crystal of compound of Formula I.

FIG. 2 is an oak ridge thermal ellipsoid plot (ORTEP, ellipsoid probability 50%) of crystal of compound of Formula I.

FIG. 3 is a molecular configuration diagram of crystal of compound of Formula I (solvent molecules, hydrogen atoms on achiral atoms omitted).

FIG. 4 is a dimer structure of crystal of compound of Formula I.

FIG. 5 is a three-dimensional stacking structure of crystal of compound of Formula I.

FIG. 6 is a DSC pattern of crystal of compound of Formula I.

FIG. 7 is an infrared spectrum of crystal of compound of Formula I.

FIG. 8 is a XRPD pattern of amorphous form of ruxolitinib.

FIG. 9 is an infrared spectrum of amorphous form of ruxolitinib.

FIG. 10 is a DSC pattern of amorphous form of ruxolitinib.

FIG. 11(a) is a XRPD pattern of crystal of compound of Formula I at 0 months, and FIG. 11(b) is the XRPD pattern of the crystal after 3 months at 30 to 35.

### SPECIFIC MODE FOR CARRYING OUT THE INVENTION

The method of the invention is further illustrated by the following examples. It should be understood that the following examples are provided only for the purpose of enabling a better understanding of the invention and are not intended to limit the scope of the invention in any way.

The raw material of ruxolitinib free base compound used was prepared according to the method of patent WO2007070514A1. The organic solvent reagents used, such as methanol, ethanol, isopropanol, ethyl acetate, isopropyl acetate, hexane, n-heptane, citric acid, etc., are commercially available.

### Preparation of Compound of Formula I

### EXAMPLE 1

2 L reaction flask was charged with 85.1 g of ruxolitinib foam (86.5% purity of (R)-ruxolitinib), 297.5 g of isopropanol. The temperature was raised to 25 to 30° C. It was stirred to dissolve. The internal temperature in the flask was controlled to 25 to 30° C. 425.0 g of purified water was added dropwise. After adding, it was turbid in the flask. The internal temperature was controlled to 25 to 30° C., and it was stirred for 1 to 3 hours. After a large amount of solids were precipitated, it was cooled to 0-10° C. and kept this temperature and stirred for 1 to 2 hours, filtered, rinsed with a small amount of 41% isopropanol aqueous solution at 0-10° C., filtered and dried. It was dried under vacuum at 40-45° C. for 4 to 7 hours, to obtain 74.9 g of compound of Formula I and the purity of (R)-ruxolitinib was 99.8%.

### EXAMPLE 2

2 L reaction flask was charged with 90.0 g of ruxolitinib oil (85.3% purity of (R)-ruxolitinib) and 270.0 g of ethanol. The temperature was raised to 30 to 40° C. It was stirred to dissolve. The internal temperature in the flask was controlled to 30 to 40° C. 630.0 g of purified water was added dropwise. After adding, it was turbid in the flask. It was stirred for 1 to 3 h. After a large amount of solids were precipitated, it was cooled to -5 to 5° C., and kept this temperature and stirred for 1 to 2 hours, filtered, rinsed with a small amount of 30% ethanol aqueous solution at −5 to 5° C., filtered and dried. It was dried under vacuum at 35 to 40° C. for 5 to 8 hours, to obtain 77.0 g of compound of Formula I and the purity of (R)-ruxolitinib was 99.0%.

### EXAMPLE 3

500 mL reaction flask was charged with 45.0 g of ruxolitinib oil (85.3% purity of (R)-ruxolitinib) and 80.0 g of tetrahydrofuran. The temperature was raised to 20 to 30° C. It was stirred to dissolve. The internal temperature in the flask was controlled to 20 to 30° C. 120 g of purified water was added dropwise. After adding, it was turbid in the flask. 0.1 g of compound of formula I obtained from the above Example was added as crystal seeds. The internal temperature was controlled to 20 to 30° C. and stirred for 1 to 2 hours. After a large amount of solids were precipitated, it was cooled to −2 to 8° C., and kept this temperature and stirred for 1 to 2 hours, filtered, rinsed with a small amount of 40% tetrahydrofuran aqueous solution at −2 to 8° C., filtered and dried. It was dried under vacuum at 40 to 45° C. for 6 to 9 hours, to obtain 36.7 g of compound of Formula I and the purity of (R)-ruxolitinib was 98.6%.

### EXAMPLE 4

1 L reaction flask was charged with 45.0 g of free base amorphous form (86.5% purity of (R)-ruxolitinib) and 70.0 g of menthol. The temperature was raised to 25 to 35° C. It was stirred to dissolve. The internal temperature in the flask was controlled to 25 to 35° C. 180 g of purified water was added dropwise. After adding, it was turbid in the flask. 0.1 g of compound of formula I obtained from the above Example was added as crystal seeds. The internal temperature was controlled to 25 to 35° C. and stirred for 1 to 2 hours. After a large amount of off-white solids were precipitated, it was cooled to 0 to 5° C., and kept this temperature and stirred for 1 to 2 hours, filtered, rinsed with a small amount of 28% menthol aqueous solution at 0 to 5° C., filtered and dried. It was dried under vacuum at 35 to 45° C. for 4 to 7 hours, to obtain 38.7 g of compound of Formula I and the purity of (R)-ruxolitinib was 99.4%.

### Preparation of Amorphous Ruxolitinib

### EXAMPLE 5

250 mL reaction flask was charged with 50.0 g of ruxolitinib oil (85.3% purity of (R)-ruxolitinib) and 75.0 g of isopropyl acetate, and stirred to dissolve for further use.

2 L reaction flask was charged with 800 g of n-heptane and cooled to −5 to 5° C., followed by the addition dropwise of the above solution of ruxolitinib in isopropyl acetate. After adding, the internal temperature was controlled to 0-5° C., stirred and crystallized for 2 to 4 hours, a large amount of yellowish solid was precipitated, and filtered. The filter cake was washed with a small amount of 8.6% isopropyl acetate and n-heptane solution, filtered and dried. It was dried under vacuum at 30 to 40° C. for 5 to 6 hours, to obtain 45.8 g of amorphous ruxolitinib and the purity of (R)-ruxolitinib was 91.3%.

### EXAMPLE 6

The temperature of 70.0 g of compound of formula I (99.8% purity of (R)-ruxolitinib) was controlled to 50-55° C., and the vacuum degree was ≤−0.09 Mpa. It was dried for 10 to 12 hours, to obtain 82.3 g of amorphous ruxolitinib and the purity of (R)-ruxolitinib was 99.8%.

### EXAMPLE 7: THE PREPARATION OF RUXOLITINIB PHOSPHATE

In a 150 mL reaction flask, 30.7 g of ruxolitinib oil (99.0% purity of (R)-ruxolitinib) and 11.3 g of phosphoric acid were added, followed by 30 g of isopropanol under the protection of nitrogen. After heating to dissolve, it was crystallized under naturally cooling, stirred at 5 to 10° C. for 0.5 to 1.0 hours, filtered, and dried under vacuum at 50 to 55° C. for 5 to 8 hours, to obtain 35.7 g of ruxolitinib phosphate (the purity of (R)-ruxolitinib was 99.0%).

### EXAMPLE 8: THE STABILITY OF SOLID FORMS

3.0 g of ruxolitinib free base in different solid forms was stored in a stabilization chamber, and placed in the condition that the temperature and humidity were controlled to at 25° C., 60% RH, and 40° C., 75% RH for 2 and 8 hours, respectively. The samples were analyzed by appearance or XRPD to check its solid form and compared with its initial solid form. According to the results shown in Table 4, the compound of formula I (dihydrate crystal) prepared in Examples 1 and 4 show better stability than ruxolitinib foam (prepared according to the method of Organic Letters, 2009, 11(9), 1999-2002) and amorphous form prepared in Example 5.

TABLE 4
Stability of different solid forms of ruxolitinib free base

| | Physical stability | | |
|---|---|---|---|
| Samples | Starting | 25° C./60%-RH, 2 hours | 40° C./75%-RH, 8 hours |
| Compound of Formula I of | Dihydrate | No change of solid form | No change of solid form |
| Example 1 | | | |
| Compound of Formula I of | Dihydrate | No change of solid form | No change of solid form |
| Example 4 | | | |
| Foam | Amorphous | Change of solid form, | Change of solid form, |
| | form | hygroscopic | hygroscopic |
| Amorphous form of | Amorphous | Change of solid form, | Change of solid form, |
| Example 5 | form | hygroscopic | hygroscopic |

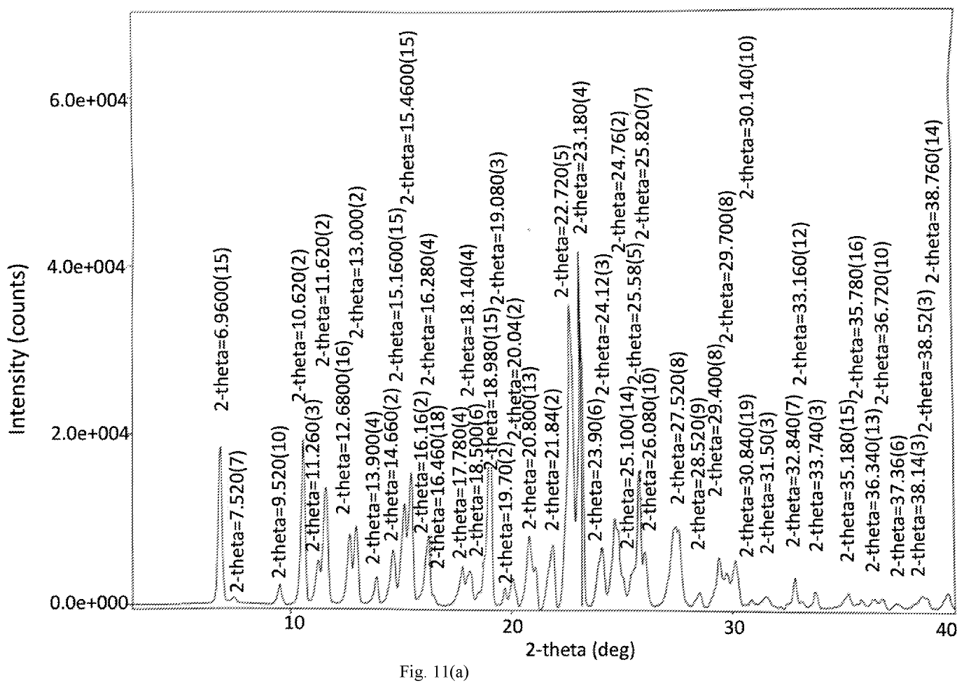

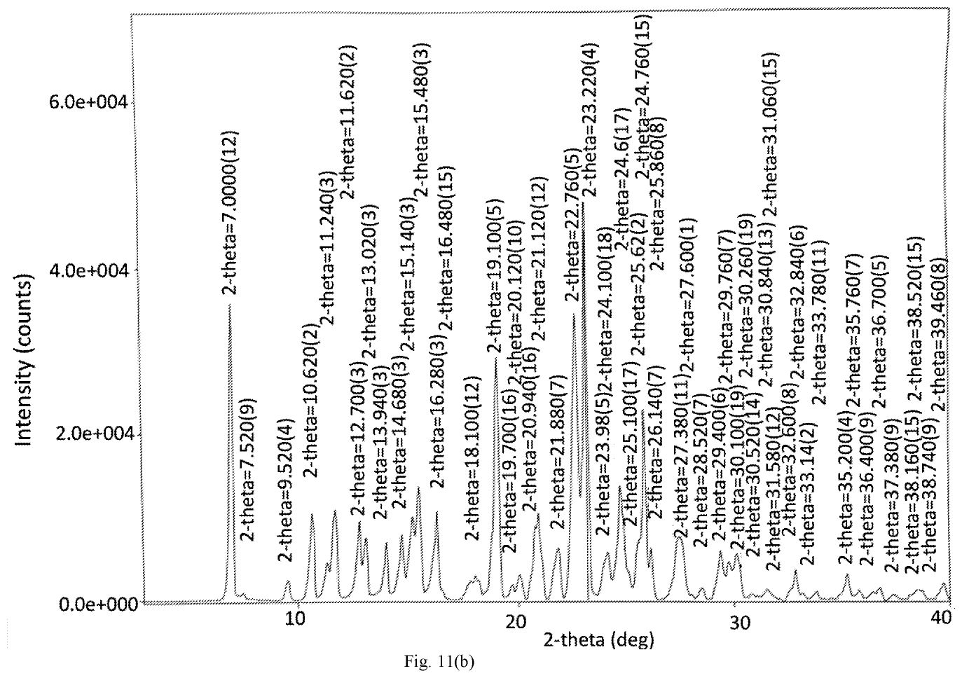

In addition, 10.0 g of crystal of compound of formula I of Example 1 was placed in a transparent polyethylene self-sealing bag and allowed to stand at 30-35° C. for 3 months. The XRPD pattern shows that the crystalline form remains stable as shown in FIGS. 11(a) and (b).

1. A compound of ruxolitinib dihydrate of Formula I, 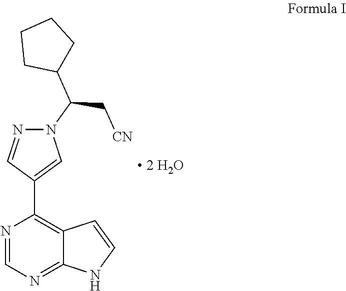 [SMILES: N#CC[C@H](C1CCCC1)n1cc(-c2ncnc3[nH]ccc23)cn1 ; "· 2 H2O"]
which is in crystalline form and characterized in that its X-ray powder diffraction pattern has characteristic diffraction peaks expressed in 2θ diffraction angle at 6.92±0.2°, 19.02±0.2°, 22.62±0.2°, 23.12±0.2° and 24.66±0.2°.

2. The compound according to claim 1, characterized in that its X-ray powder diffraction pattern has characteristic diffraction peaks expressed in 2θ diffraction angle at 6.92±0.2°, 10.54±0.2°, 11.54±0.2°, 15.42±0.2°, 19.02±0.2°, 22.62±0.2°, 23.12±0.2° and 24.66±0.2°.

3. The compound according to claim 1, characterized in that its X-ray powder diffraction pattern has characteristic diffraction peaks expressed in 2θ diffraction angle at 6.92±0.2°, 10.54±0.2°, 11.54±0.2°, 15.42±0.2°, 19.02±0.2°, 20.78±0.2°, 22.62±0.2°, 23.12±0.2°, 24.66±0.2° and 25.76±0.2°.

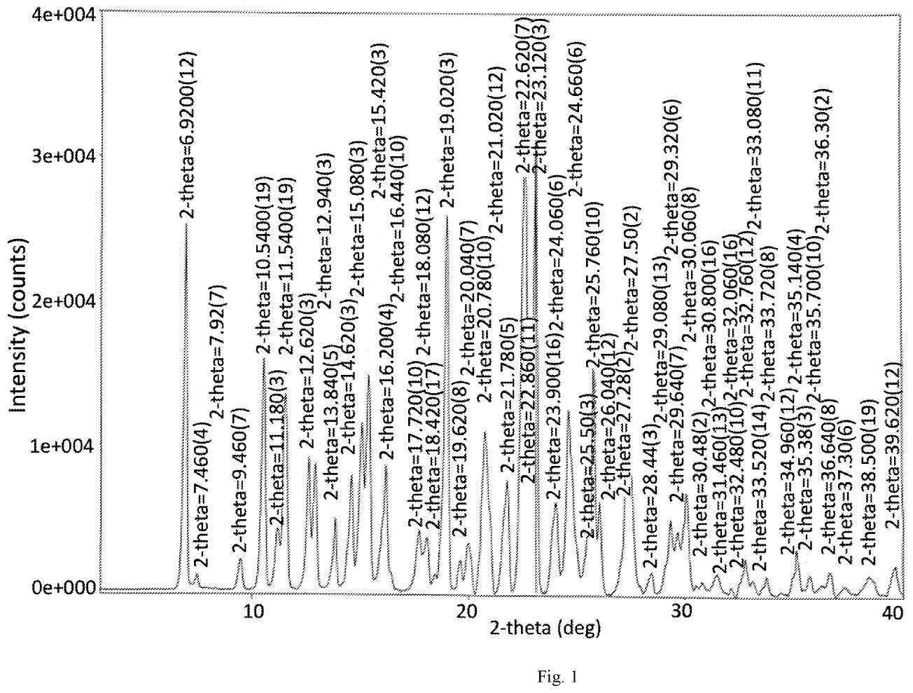

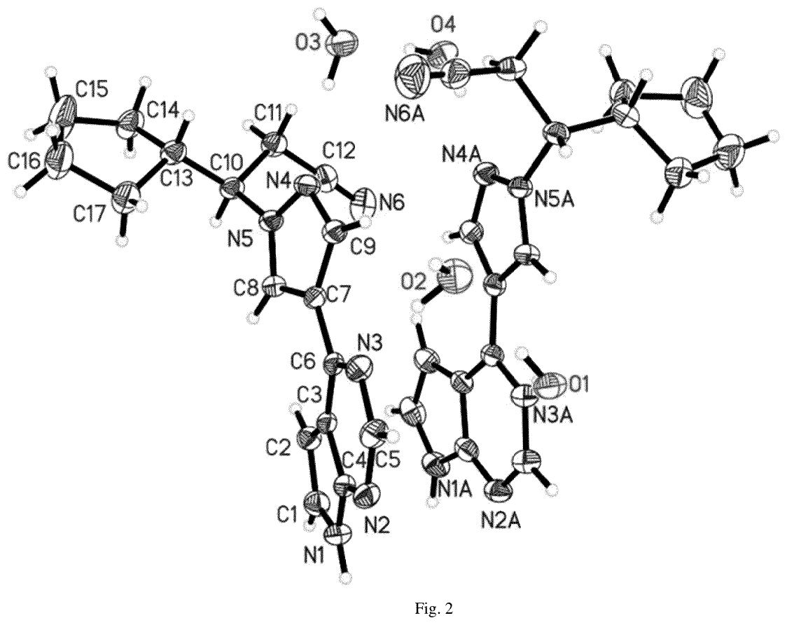

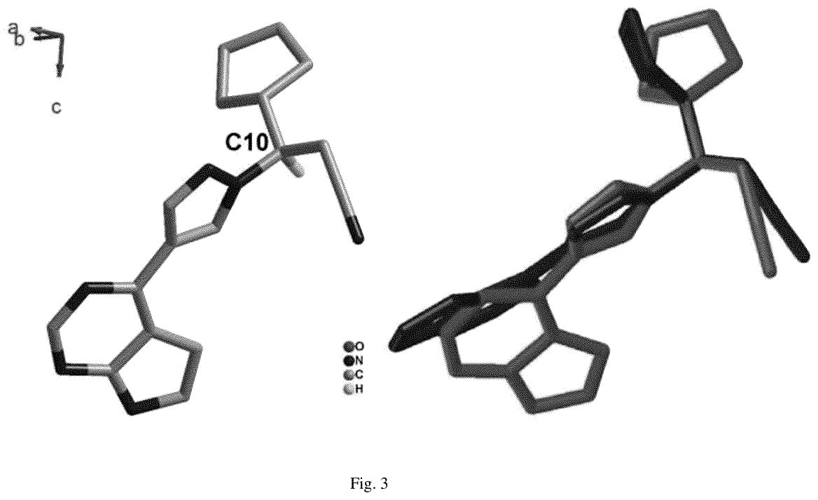

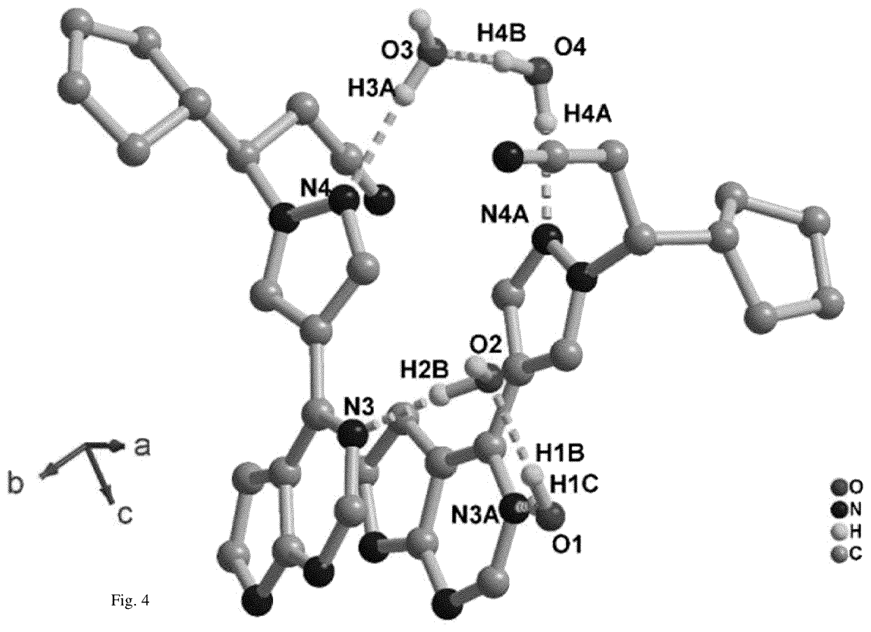

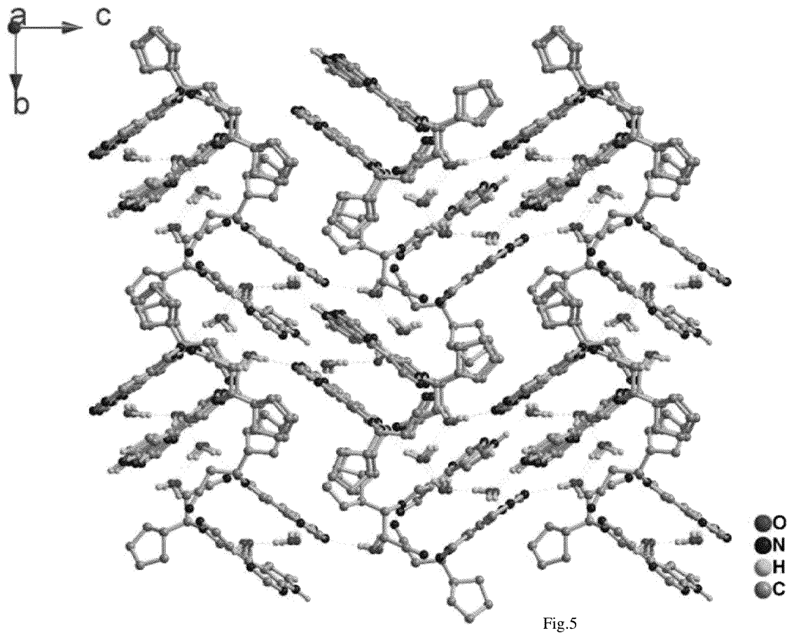

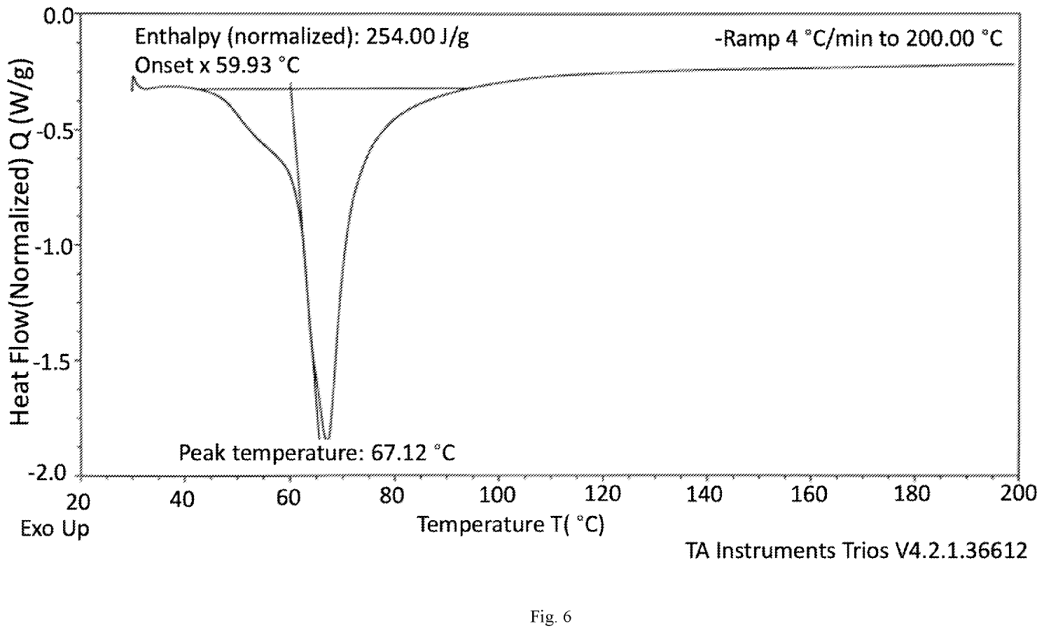

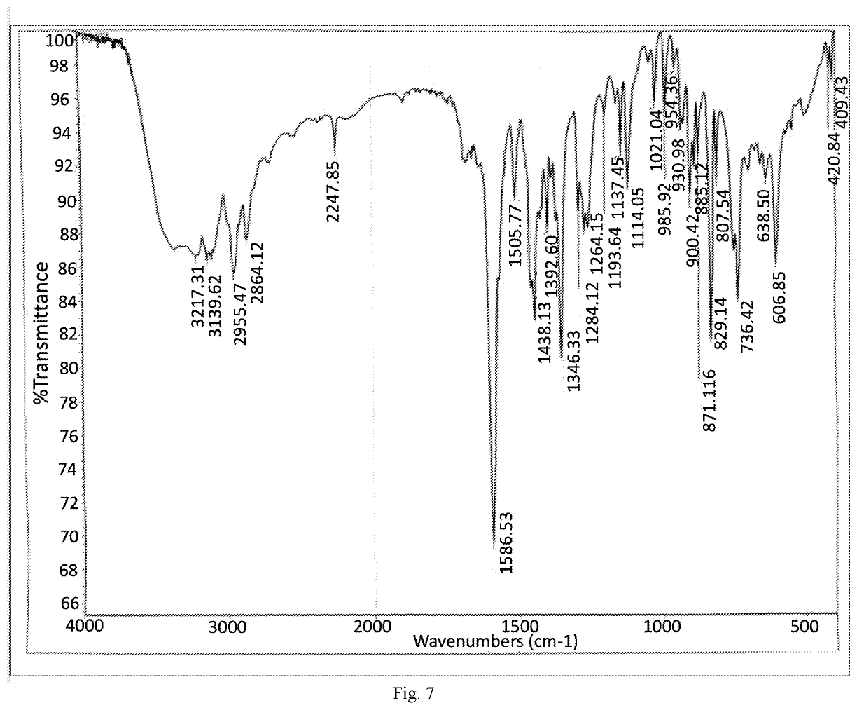

4. The compound according to claim 1, having an X-ray powder diffraction pattern as shown in FIG. 1.

5. A preparation method of the compound according to claim 1, comprising: ruxolitinib free base is dissolved in an organic solvent, then purified water is added dropwise to crystallize to obtain the crystal of compound of formula I.

6. The preparation method according to claim 5, wherein the organic solvent is selected from methanol, ethanol, n-propanol, isopropanol, n-butanol, acetone, diethyl ether, isopropyl ether, methyl tert-butyl ether, tetrahydrofuran, 1,4-dioxane, acetonitrile, dichloromethane, chloroform, toluene, chlorobenzene, hexane, heptane, DMF, DMAC, DMSO, NMP, methyl acetate, ethyl acetate, isopropyl acetate, or a mixture of two or more thereof.

7. A preparation method of amorphous ruxolitinib free base, comprising the following steps: ruxolitinib free base raw material is added in an organic solvent and dissolved, antisolvent is added to crystallize, to obtain amorphous form of ruxolitinib free base in solid.

8. The preparation method according to claim 7, wherein the organic solvent is selected from ethyl formate, methyl acetate, ethyl acetate, isopropyl acetate, diethyl ether, isopropyl ether, methyl tert-butyl ether, tetrahydrofuran, 1,4-dioxane, acetone, dichloromethane, toluene, xylene, chlorobenzene, or mixtures of two or more thereof.

9. The preparation method according to claim 7, wherein the antisolvent is selected from pentane, n-pentane, neopentane, hexane, n-hexane, cyclohexane, methylcyclohexane, n-heptane, or a mixture of two or more thereof.

10. A preparation method of amorphous form of ruxolitinib free base, comprising: compound of formula I according to claim 1 is dried under vacuum to lose crystal water, to obtain amorphous form of ruxolitinib free base.

11. The preparation method according to claim 10, wherein the conditions of drying under vacuum are as follows:
vacuum degree is ≤−0.1 Mpa; and
drying temperature is 30 to 65° C., preferably 35 to 60° C., and most preferably 45 to 55° C.

12. Ruxolitinib free base in amorphous form prepared by the preparation method according to claim 7.

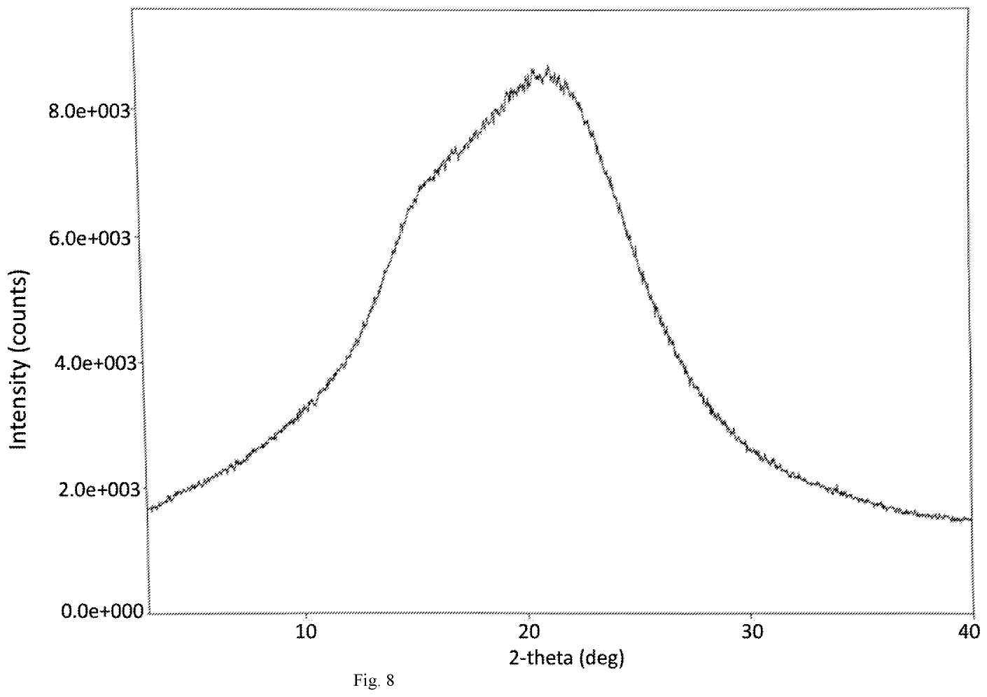

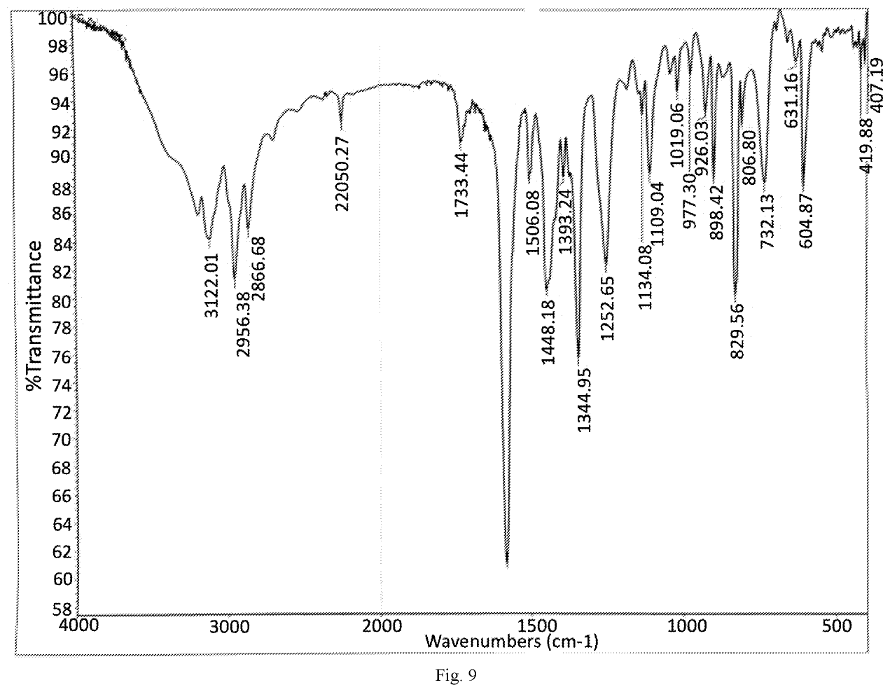

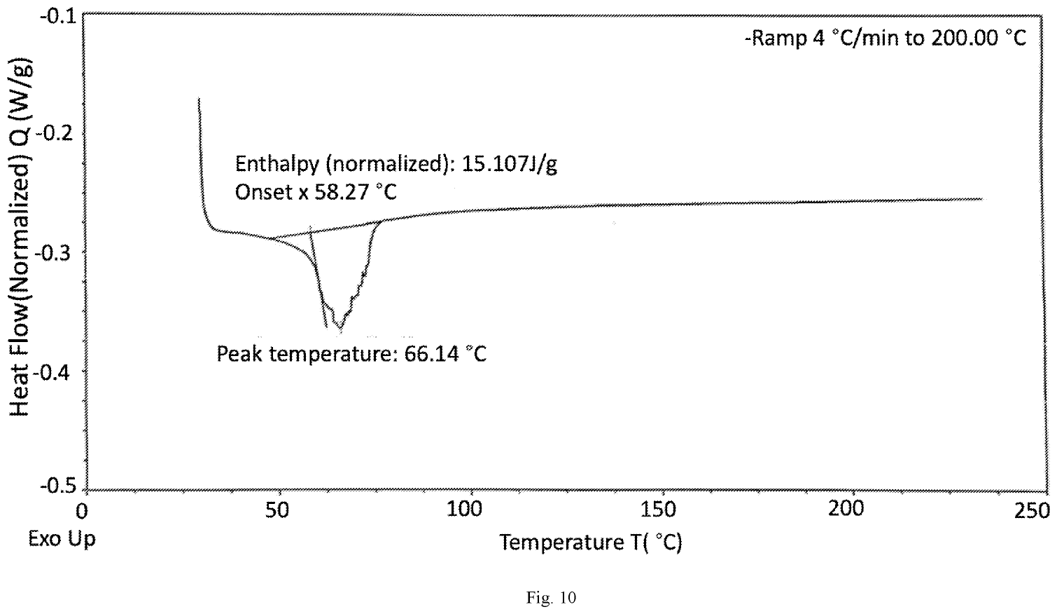

13. Ruxolitinib free base in amorphous form according to claim 12, having an X-ray powder diffraction pattern as shown in FIG. 8.

14. A pharmaceutical composition comprising crystal of compound of formula I according to claim 1 and one or more pharmaceutically acceptable excipients or carriers.

15. The pharmaceutical composition according to claim 14, comprising ruxolitinib, an organic acid and other excipients or carriers, wherein the organic acid is selected from one or more of malonic acid, maleic acid, fumaric acid, citric acid, tartaric acid and malic acid.

16. Ruxolitinib free base in amorphous form prepared by the preparation method according to claim 10.

17. Ruxolitinib free base in amorphous form according to claim 16, having an X-ray powder diffraction pattern as shown in FIG. 8.

18. A pharmaceutical composition comprising ruxolitinib free base in amorphous form according to claim 12 and one or more pharmaceutically acceptable excipients or carriers.

19. A pharmaceutical composition comprising ruxolitinib free base in amorphous form according to claim 13 and one or more pharmaceutically acceptable excipients or carriers.

20. The pharmaceutical composition according to claim 18, comprising ruxolitinib, an organic acid and other excipients or carriers, wherein the organic acid is selected from one or more of malonic acid, maleic acid, fumaric acid, citric acid, tartaric acid and malic acid.

21. The pharmaceutical composition according to claim 19, comprising ruxolitinib, an organic acid and other excipients or carriers, wherein the organic acid is selected from one or more of malonic acid, maleic acid, fumaric acid, citric acid, tartaric acid and malic acid.
